(19) United States
(12) Patent Application Publication
(10) Pub. No.: US 2012/0310370 A1
(43) Pub. Date: Dec. 6, 2012

(54) SYSTEMS AND METHODS FOR PROVIDING A NEURAL-MACHINE INTERFACE FOR ARTIFICIAL LEGS

(75) Inventors: He Huang, Lincoln, RI (US); Qing K. Yang, Saunderstown, RI (US); Yan Sun, Wakefield, RI (US)

(73) Assignee: The Board of Governors for Higher Education, State of Rhode Island and Providence Plantations, Providence, RI (US)

(21) Appl. No.: 13/556,527

(22) Filed: Jul. 24, 2012

Related U.S. Application Data
(63) Continuation of application No. PCT/US11/22349, filed on Jan. 25, 2011.
(60) Provisional application No. 61/297,960, filed on Jan. 25, 2010.

(51) Int. Cl. A61F 2/72

(52) U.S. Cl. 623/25

(57) ABSTRACT

A neural-machine interface system is disclosed for providing control of a leg prosthesis. The system includes a plurality of input channels for receiving electromyographic signals from a subject, a feature vector formation unit for processing the electromyographic signals, and a pattern classification unit for identifying the intended movement of the subject's leg prosthesis.

### PRIORITY

The present application claims priority to U.S. Provisional Patent Application Ser. No. 61/297,960 filed Jan. 25, 2010, the entire disclosure of which is hereby incorporated by reference.

### GOVERNMENT SPONSORSHIP

The present invention was developed, in part, with assistance from the United States Government under National Science Foundation Grant No. 0931820. The United States government has certain rights to this invention.

### BACKGROUND

The present invention generally relates to prosthesis systems, and relates in particular to lower-limb prosthesis systems for leg amputees.

There are over 32 million amputees worldwide whose lives are severely impacted by the loss of a limb, and this number is expected to continue to grow as the population ages and as the incidence of dysvascular disease increases. Over 75% of major amputations were lower-limb, with nearly 17% of lower-limb amputees suffering bilateral amputations. There is a continued need therefore, to provide this large and growing population of amputees with the best care and return of function as possible. With the rapid advances of cyber system technologies, it has been witnessed in recent years that high speed, low cost, and real time embedded computers are widely applied in biomedical systems. The use of computerized prosthetic legs is one prominent example, in which motion and force sensors as well as a microcontroller embedded in the prosthesis form a close loop control and allow the user to produce natural gait patterns. The function of such a computerized prosthesis however, is still limited. The relatively primitive prosthesis control is based entirely on mechanical sensing without the knowledge of user intent. Users have to input to the prostheses their intended activities manually or using body motion, which is cumbersome and does not allow smooth task transitions. The fundamental limitation on all existing prosthetic legs is lack of neural control that would allow the artificial legs to move naturally as if they were his/her own limb.

Previous research has shown that some systems that use electromyographic (EMG) signals for controlling artificial upper limbs have been clinically successful, but no EMG-controlled lower limb prosthesis is currently available. It is believed that the following technical challenges exist in trying to provide EMG-controlled systems to lower limbs prostheses

First, in human physiological systems, EMG signals recorded from leg muscles during dynamic movements are highly non-stationary. Dynamic signal processing strategies are required for accurate decoding of user intent from such signals. In addition, patients with leg amputations may not have enough EMG recording sites available for neuromuscular information extraction due to the muscle loss. Maximally extracting neural information from such limited signal sources is necessary.

A second important challenge is that the accuracy in identifying the user's intent for artificial legs is more critical than that for upper limb prostheses. A 90% accuracy rate might be acceptable for control of artificial arms, but it may result in one stumble out of ten steps when used with a lower limb prosthesis, which is clearly inadequate for safe use of artificial legs. Achieving high accuracy is further complicated by environmental uncertainty, such as perspiration, temperature change, and movement between the residual limb and prosthetic socket may cause unexpected sensor failure, influence the recorded EMG signals, and reduce the trustworthiness of the neural-machine interface (NMI). It is critical to develop a reliable and trustworthy NMI for safe use of prosthetic legs.

A third challenge is to provide the compact and efficient integration of software and hardware in an embedded computer system in order to make an EMG-based NMI practical and available to patients with leg amputations. Such an embedded system would have to provide high speed and real time computation of neural deciphering algorithm because any delayed decision-making from the NMI also introduces instability and unsafe use of prostheses. Streaming and storing multiple sensor data, deciphering user intent, and running sensor monitoring algorithms at the same time superimpose a great challenge to the design of an embedded system for the NMI of artificial legs.

There remains a need, therefore, for a lower-limb prosthesis control system that provides amputees with the best care and return of function as possible.

### SUMMARY

In accordance with an embodiment, the invention provides a neural-machine interface system for providing control of a leg prosthesis. The system includes a plurality of input channels for receiving electromyographic signals from a subject, a feature vector formation unit for processing the electromyographic signals, and a pattern classification unit for identifying the intended movement of the subject's leg prosthesis.

In accordance with a further embodiment, the invention provides a neural-machine interface system for providing control of a lower limb. The system includes a plurality of input channels for receiving a plurality of sensor output signal from a subject, a processing unit for processing the plurality of sensor output signals, a pattern classification unit for identifying the intended movement of a subject's leg, and a sensor trust evaluation unit for providing a trust valuation representative of the reliability of each of the plurality of sensor output signals.

In accordance with a further embodiment, the invention provides a method of providing control of a leg prosthesis wherein the method includes the steps of receiving a plurality of electromyographic signals at a plurality of input channels, processing the plurality of electromyographic signals, and, identifying the intended movement of a subject's leg prosthesis.

### BRIEF DESCRIPTION OF THE DRAWINGS

The following description may be further understood with reference to the accompanying drawings in which:

FIG. 1 shows an illustrative diagrammatic view of the architecture of a neural-machine interface in accordance with an embodiment of the invention;

FIGS. 2A and 2B show illustrative flowcharts of a procedure for recoding feature vectors associated with different motions, and of a procedure for using the system respectively in accordance with an embodiment of the invention;

FIG. 3 shows an illustrative flowchart of a disturbance detection procedure for each sensor in accordance with an embodiment of the invention;

FIG. 4 shows an illustrative flowchart of a trust management procedure in accordance with an embodiment of the invention;

FIG. 5 shows an illustrative diagrammatic view of hardware architecture for use in a system in accordance with an embodiment of the invention;

FIG. 6 shows an illustrative block diagram of an embedded design of a system in accordance with an embodiment of the invention;

FIG. 7 shows an illustrative timing control diagram of a decision making process in accordance with an embodiment of the invention;

FIG. 8 shows an illustrative representation of response data over a time period for a motion (standing/sitting) wherein the motion changes overly the response data for the time period; and

FIGS. 9A-9C show illustrative timing diagrams of EMG signal amplitude, detection results, and trust value data respectively in a system in accordance with an embodiment of the invention.

The drawings are shown for illustrative purposes only.

### DETAILED DESCRIPTION

Applicants have discovered that the quality of life of leg amputees may be improved dramatically by using a cyber physical system (CPS) that controls artificial legs based on neural signals representing amputees' intended movements. The key to the CPS system is the neural-machine interface (NMI) that senses electromyographic (EMG) signals to make control decisions. The present application presents a design and implementation of an NMI using an embedded computer system to collect neural signals from a physical system—a leg amputee, provide adequate computational capability to interpret such signals, and make decisions to identify user's intent for prostheses control in real time. A deciphering algorithm, composed of an EMG pattern classifier and a post-processing scheme, was also developed to identify the user's intended lower limb movements.

A trust management mechanism was also designed to account for environmental uncertainty and to handle unexpected sensor failures and signal disturbances. Integrating the neural deciphering algorithm with the trust management mechanism resulted in a highly accurate and reliable software system for neural control of artificial legs. The software was then embedded in a newly designed hardware platform based on an embedded microcontroller and a graphic processing unit (GPU) to form a complete NMI for real time applications. Real time experiments on a leg amputee subject and an able-bodied subject have been successfully carried out to test the control accuracy of the new NMI.

To address the above discussed challenges with regard to using EMG signals for controlling a lower-limb prosthesis, a neural interfacing algorithm was developed that takes EMG inputs from multiple EMG electrodes mounted on user's lower limb, decodes the user's intended lower limb movements, and monitors sensor behaviors based on trust models as discussed further below. The EMG pattern recognition (PR) algorithm together with a post-processing scheme effectively process non-stationary EMG signals of leg muscles, for accurately deciphering user intent. The neural deciphering algorithm consists of two phases: offline training and online testing. To ensure the trustworthiness of NMI under uncertain environmental conditions, a real time trust management (TM) module was designed and implemented to examine the changes of the EMG signals and estimate the trust level of individual sensors. The trust information may be used to reduce the impact of untrustworthy sensors on the system performance.

The deciphering algorithm was implemented on an embedded hardware architecture as an integrated NMI to be carried by leg amputees. Two key requirements for the hardware architecture were high speed processing of training processes and real time processing of the interfacing algorithm. To meet these requirements, the embedded architecture consisted of an embedded microcontroller, a flash memory, and a graphic processing unit (GPU). The embedded microcontroller provided necessary interfaces for analog to digital (A/D) and digital to analog (D/A) signal conversion and processing and computation power needed for real time control. The control algorithm was implemented on the bare machine with memory and IO managements without using the existing OS to avoid any unpredictability and variable delays. The flash memory was used to store training data. The EMG PR training process involved intensive signal processing and numerical computations, which needs to be done periodically when the system trust value is low. Such computations may be done efficiently using modern GPUs that provide supercomputing performance with very low cost. New parallel algorithms specifically tailored to the multi-core GPU were developed exploiting memory hierarchy and multithreading of the GPU. Substantial speedups of the GPU for training process were achieved making the classifier training time tolerable in practice.

A complete prototype was built implementing all the software and hardware functionalities. The prototype was used to carry out real time testing on human subjects, including a male patient with unilateral transfemoral amputations. A goal of the experiments was to use the NMI prototype to sense, collect, and decode neural muscular signals of the human subject. Based on the neural signals, the NMI tries to interpret the subject's intent for sitting and standing, two basic but difficult tasks for patients with transfemoral amputations due to the lack of power from the knee joint. The trust management module was also tested on a male able-bodied subject by introducing motion artifacts during the subject's nominal sitting and standing task transitions. The detection rate and false alarm rate for distribution detection was evaluated.

The extensive experiments of the NMI on the human subjects have shown promising results. Among the 30 sitting-to-standing transitions and the 30 standing-to-sitting transitions of the amputee subject, the NMI recognized all the intended transitions correctly with the maximum decision delay of 400 ms. The algorithm may also filter out occasional signal disturbances and motion artifacts, and has been found to have a 99.37% detection rate and a 0% false alarm rate.

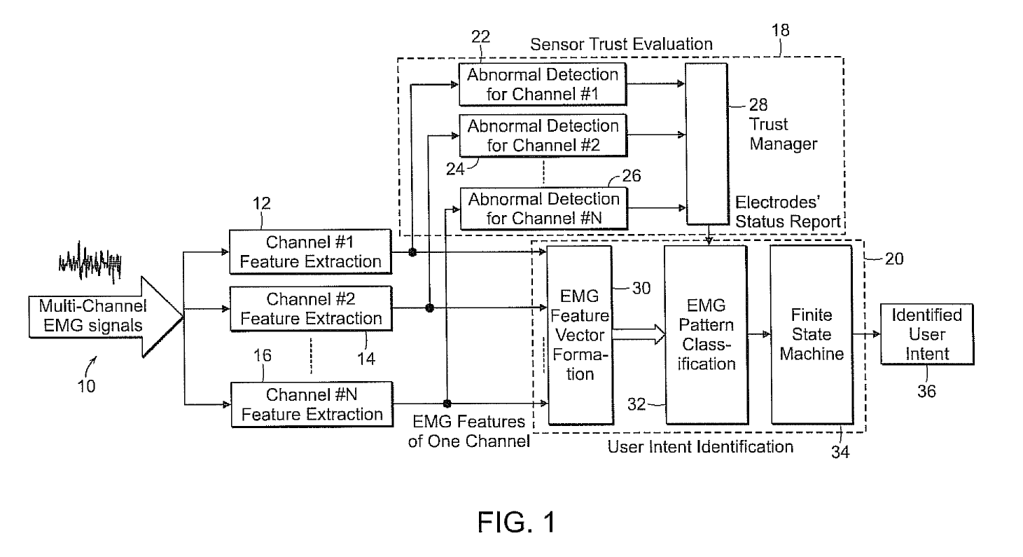

FIG. 1 shows the software architecture of a neural-machine interface system in accordance with an embodiment of the invention. The system receives EMG signals from multiple channels as shown at 10, and for each channel (e.g., 1 to N) the system extracts features as shown at 12, 14 and 16. The feature extraction is achieved by pattern recognition analysis, and for each channel, the extracted features are provided to both a sensor trust evaluation system 18 and a user intent identification system 20. As shown at 22, 24 and 26, the sensor trust evaluation system 18 determines for each channel whether the EMG signal for that channel is abnormal. An indication of whether the signals for any channels are abnormal is provided to a trust manager 28, which provides an electrode status report to the user intent identification system 20.

The EMG signals from each of the multiple channels are also provided to an EMG feature vector formation unit 30 within the user intent identification system, and the vector data for the channels is provided to an EMG pattern classification unit, which also receives the status report from the trust evaluation system 18. The EMG pattern classification unit communicates with a finite state machine 34, which having taken into consideration any channels having been identified as not trustworthy, identifies a user' intent as shown at 36.

The multiple channels of EMG signals are therefore, the system inputs. EMG signals are preprocessed and segmented by sliding analysis windows. EMG features that characterize individual EMG signals are extracted for each analysis window. One of the two major data pathways classifies user movement intent and the other performs sensor trust evaluation as discussed above.

To identify user intent, EMG features of individual channels are concatenated into one feature vector. The goal of pattern recognition is to discriminate among desired classes of limb movement based on the assumption that patterns of EMG features at each location is repeatable for a given motion but different between motions. The output decision stream of the EMG pattern classifier is further processed to eliminate erroneous task transitions. In the path for sensor trust evaluation, the behaviors of individual sensors are closely monitored by the abnormal sensor detection units 22, 24, 26. The trust manager 28 evaluates the trust level of each sensor and then adjusts the operation of the classifier for reliable EMG pattern recognition.

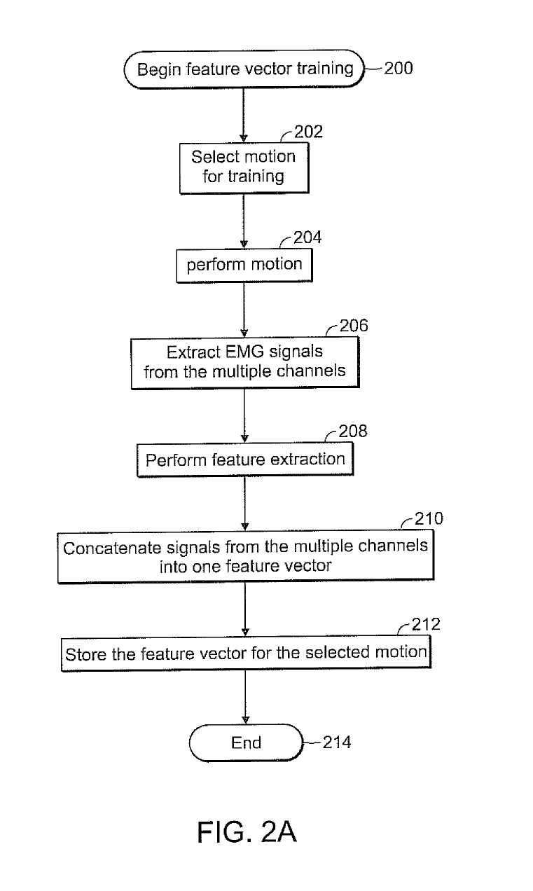

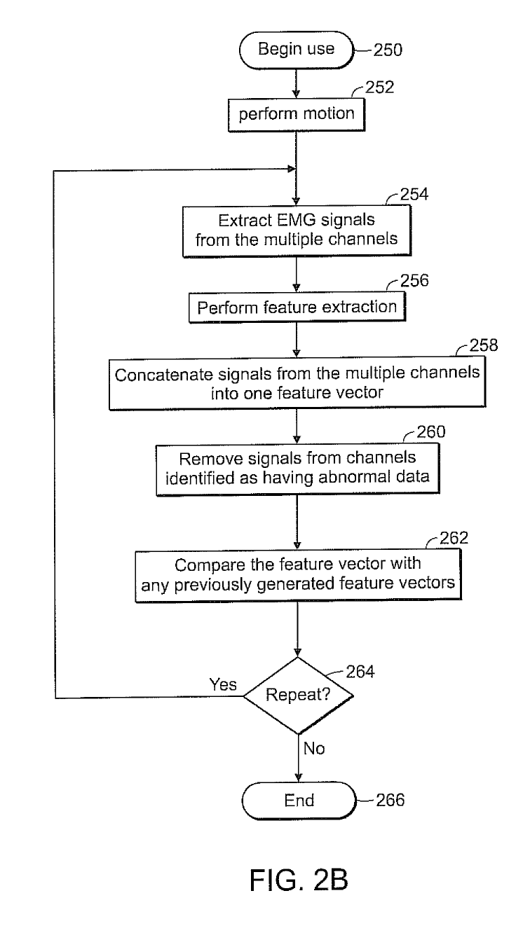

With reference to FIG. 2A, the expected feature vector for each of a plurality of motions (such as standing, sitting, ascending stairs, descending stairs etc.) may be pre-recorded by a process that begins (step 200) and a user enters a particular motion such as standing, sitting, ascending stairs or descending stairs (step 202). The user then performs the selected motion (step 204), and the system then extracts EMG signals from each of the multiple channels (step 206) and performs the feature extraction from the EMG signals (step 208). The system then concatenates the signals from the multiple channels into one feature vector (step 210) and then system then stores the current weighted average feature vector for that selected motion (step 212) and then ends (step 214).

During use of the system, and with reference to FIG. 2B, the process begins (step 250) and a user performs a motion (step 252). The system then extracts EMG signals from each of the multiple channels (step 254) and performs a feature extraction (step 256) on the EMG signals. The system then concatenates the signals from the multiple channels into one feature vector (step 258) and removes signals from channels that have been identified as having abnormal data (step 260). The process may then compare the current feature vector with any previously recorded feature vectors (step 262), and then repeat (step 264) as long as desired prior to ending (step 266).

The dynamic EMG pattern classification strategy and post-processing methods discussed above were developed for high decision accuracy. The EMG signals were recorded from gluteal and thigh muscles of a residual limb. Four time-domain (TD) features (the mean absolute value, the number of zero-crossings, the waveform length, and the number of slope sign changes) were selected for real-time operation because of their low computational complexity compared to frequency or time-frequency domain features. A linear discriminant analysis (LDA) classifier (see A new strategy for multifunction myoelectric control by B. Hudgins, P. Parker, and R. N. Scott, IEEE Transactions in Biomedical Engineering, v. 40, no. 1, pp. 82-94 (1993)) was used in the current embodiment due to the comparable classification accuracy to more complex classifiers and the computation efficiency for real-time prosthesis control. In accordance with further embodiments, various other classification methods may be employed, such as multilayer perceptron (see Classification of EMG signals using PCA and FFT by N. F. Guler and S. Kocer, Journal of Medical Systems, v. 29, no. 3, pp. 241-250 (2005)), Fuzzy logic (see A heuristic fuzzy logic approach to EMG pattern recognition for multifunctional prosthesis control by A. B. Ajiboye, and R. F. Weir, IEEE Transactions in Neural Systems Rehabilitation Engineering, v. 13, no. 3, pp. 280-291 (2005)), and artificial neural network (see A strategy for identifying locomotion modes using sur face electromyography, by H. Huang, T. A. Kuiken and R. D. Lipschutz, IEEE Transactions in Biomedical Engineering, v. 56, no. 1, pp. 65-73 (2009), the disclosure of which is hereby incorporated by reference in its entirety).

When EMG signals are non-stationary, the EMG features across time show large variation within the same task mode, which results in overlaps of features among classes and therefore low accuracy for pattern recognition. By assuming that the pattern of non-stationary EMGs has small variation in a short-time window and that EMG patterns are repeatable for each defined short-time phase, a phase-dependent EMG classifier was designed, which was successfully applied to accurately and responsively recognize the user's locomotion modes. For non-locomotion modes such as sitting and standing, the classifier can be built into the movement initiation phase by the same design concept. The structure of such a dynamic design of the classifier can be found elsewhere.

Erroneous decisions were removed from the classifier by use of a majority vote process by which the decision error was removed by smoothing the decision output. This method may further increase the accuracy of NMI, but may also sacrifice the system response time.

As mentioned above, the NMI for artificial legs must be reliable and trusted by the prosthesis users. The design goals of a trustworthy sensor system are (1) prompt and accurate detection of disturbances in real time applications, and (2) assessment of reliability of a sensor/system with potential disturbances. To achieve these goals, the system was designed to include a trust management module that contains three parts: abnormal detection, trust manager, and decision support.

As shown at 22, 24 and 26 in FIG. 1, an abnormal detector is applied to each EMG channel to detect disturbances occurring in each EMG signal. Disturbances that cause sensor malfunctions can be diverse and unexpected. Among all these disturbances, motion artifacts can cause large damage and are extremely difficult to be totally removed. Motion artifacts are also fairly common in both laboratory environments and in real-world applications. To detect abnormalities in EMG signals, a change detector was employed that identifies changes in the statistics of EMG signals. In particular, changes in two time-domain (TD) features are monitored: mean absolute value (Feₘₑₐₙ) and the number of slope sign changes (Feₛₗₒₚₑ).

Positive change in Feₘₑₐₙ and negative change in Feₛₗₒₚₑ are monitored and used as indicators of the presence of motion artifacts. Since the changes are in two directions (positive and negative), a two-sided change detector was employed.

In accordance with the present embodiment, the Cumulative Sum (CUSUM) algorithm, and in particular the two-sided CUSUM detection scheme, was employed due to its reliability in detecting small changes, its insensitivity to the probabilistic distribution of the underlying signal, and its benefit in reducing the detection delay (see Using Statistical Process Control to Monitor Active Managers, by T. Philips, E. Yashchin and D. Stein, Journal of Portfolio Management, vol. 30, no. 1, pp. 186, 191 (2003) and Continuous Inspection Scheme, by E. S. Page, Biometrika, vol. 41, no. 1/2, pp. 100-115 (1954), the disclosures of each of which are hereby incorporated by reference in their entirety).

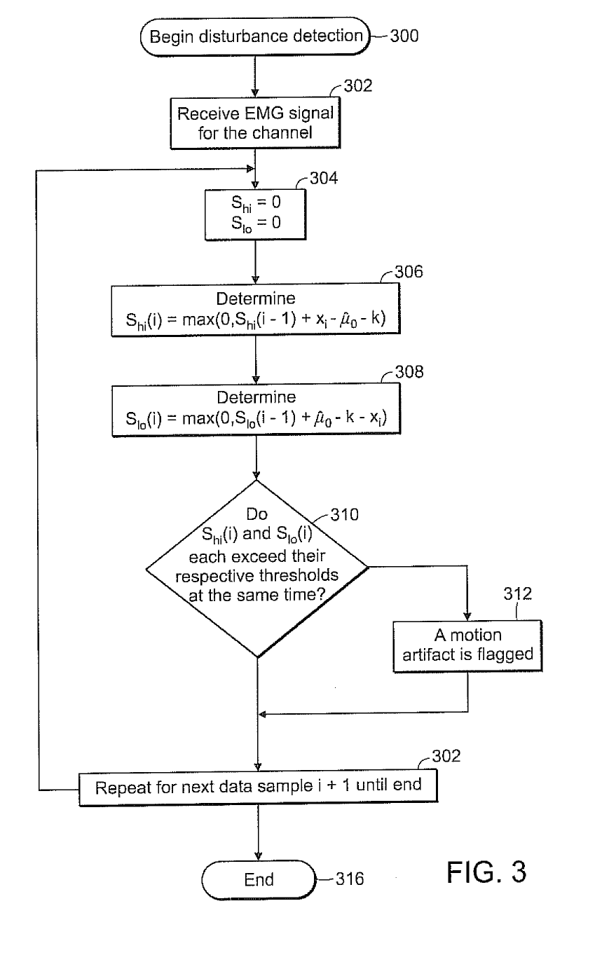

As shown in FIG. 3, the process for identifying whether a motion artifact in a channel is detected begins (step 300) by first receiving an EMG signal from the channel (step 302). The system then sets to zero the initial values Sₕᵢ and Sₗₒ for the two-sided CUSUM detector (step 304). The system then determines (steps 306 and 308) the following values:

Sₕᵢ(i)=max(0,Sₕᵢ(i−1)+xᵢ−\hat{μ}−k)

and

Sₗₒ(i)=max(0,Sₗₒ(i−1)+\hat{μ}₀−k−xᵢ)

where xᵢ represents the i^{th} data sample, \hat{μ}₀ is the mean value of data without changes, and k is the CUSUM sensitivity parameter. The smaller the value k is, the more sensitive the CUSUM detector is to small changes. In steps 306 and 308, Sₕᵢ and Sₗₒ are used for detecting the positive and negative changes, respectively. If Sₕᵢ exceeds a certain positive threshold (Thₚ), then a positive change is detected, and if Sₗₒ exceeds a certain negative threshold (Thₙ), then a negative change is detected.

The system then determines (step 310) whether both a positive change and a negative change occurred since the presence of a positive change in Feₘₑₐₙ and a negative change in Feₛₗₒₚₑ at the same time may serve as the indicator of a motion artifact in accordance with the present embodiment; in this case, a motion artifact is flagged (step 312). The value Sₕᵢ is therefore applied to detect positive changes in Feₘₑₐₙ and the value Sₗₒ is applied to detect negative changes in Feₛₗₒₚₑ. Again, when Sₕᵢ and Sₗₒ exceed their corresponding thresholds at the same time, a motion artifact is detected.

In step 306, the value xᵢ denotes the i^{th} sample of Feₘₑₐₙ and xᵢ is calculated as mean of the absolute value of EMG signal within the i^{th} window. In step 308, the value xᵢ denotes the i^{th} sample of Feₛₗₒₚₑ and is calculated as the number of the slope sign changes within the i^{th} window. The value \hat{μ} in both steps (306 and 308) is computed as the average of xᵢ before any changes were detected. The sensitivity parameter (k) is set as 0.05, and the threshold Th is set as 0.1 for both of steps 306 and 308.

In the real time testing, once the CUSUM detector detects a change, it will raise an alarm and restart (step 314) by setting Sₕᵢ and Sₗₒ to 0 again (step 304) in order to detect the next change in a new data sample. By doing so, the system can respond sensitively and promptly to multiple changes in the EMG signal prior to ending (step 316).

The CUSEM detector therefore promptly respond to disturbances, and then restarts for the next round disturbance detection right after it detects one disturbance. In certain applications, however, there may be a disturbance lasting for an extending period of time, and the CUSUM detector would then detect it for more than once. This may lead to an inaccurate trust calculation. To avoid this problem, a post processing scheme is proposed to stabilize the detection result. In this post processing scheme, the two disturbances that are very close to each other are combined (i.e., within continuous windows) as one disturbance. In the real time testing, L is set as 3, which represents 240 ms. If the detector is triggered twice within 240 ms therefore, the two disturbances are considered to be one disturbance.

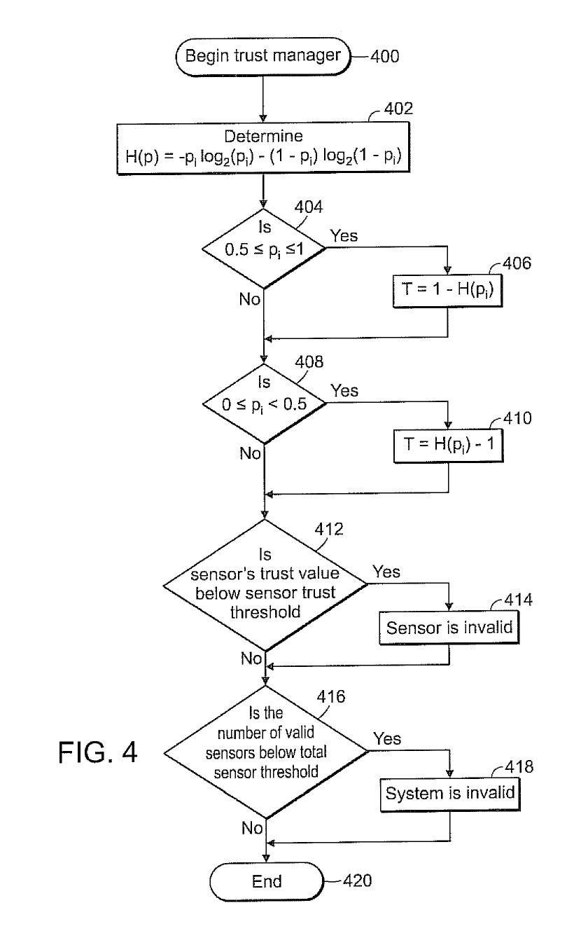

FIG. 4 shows a trust management process in accordance with an embodiment of the invention by which the system determines whether a detected disturbance (from the method of FIG. 3) represents either permanent damage in the sensor or recoverable damage in the sensor. In particular, after the abnormal detector detects the disturbance in an EMG signal, the EMG sensor is expected to be either permanently damaged or perfectly recoverable. To evaluate the trust level of the sensor, the value p₁ denotes the probability that a sensor behaves normally after one disturbance is detected.

In particular, the trust management process begins (step 400) by assuming that all disturbances are independent. The probability that a sensor is still normal after i disturbances, denoted by pᵢ=p₁ⁱ. The process then determines (step 402) an entropy value (H(pᵢ)) as

H(pᵢ)=−pᵢ log₂(pᵢ)−(1−pᵢ)log₂(1−pᵢ)

The trust value is computed from the probability value by the entropy-based trust quantification method (steps 404, 406, 408, 410), as

$$T = \{ \begin{matrix}1 - H ( {p}_{i} ) , if 0.5 \leq {p}_{i} \leq 1 \\ H ( {p}_{i} ) - 1 , if 0 \leq {p}_{i} < 0.5\end{matrix} \}$$

where T is the trust value and H(pᵢ) is the entropy (see Information Theoretical Framework of Trust Modeling and Evaluation for Ad Hoc Networks, by Y. Sun, W. Yu, Z. Han, and R. Liu, IEEE Journal on Selected Areas of Communications, v. 24, no. 2 (2006)).

Different p₁ values should be set according to the nature of the disturbance. The larger the p₁ value, the less likely the disturbance can damage the sensor. The calculation of trust is extendable to the case that different disturbances are detected for one sensor.

The trust information is provided to the user intent identification (UII) module to assist trust-based decisions, and there are therefore, two levels of decisions: 1) Sensor level, and 2) system level. If the sensor's trust value is below a sensor trust value (step 412), then the sensor is determined to be invalid (step 414). When the sensor's trust value drops below a threshold, this sensor is considered as damaged, and its reading is removed from the UII module. For example, if two disturbances, whose p₁ values are 0.8 and 0.9, respectively, are detected for a sensor, the pᵢ value may be replaced by 0.8×0.9. In the above system, the p₁ value for motion artifact was set to 0.9.

If the number of valid sensors is determined to below a total sensor threshold (step 416), then the system is determined to be invalid (step 418) and ends (step 420). After removing the damaged sensors, the system trust may be calculated by the summation of trust values of the remaining sensors. If the system trust is lower than a threshold therefore, the entire UII model is not trustworthy, and actions for system recovery must be taken. One possible action is to re-train the classifier. Another possible action is to instruct the patient to manually examine the artificial leg system.

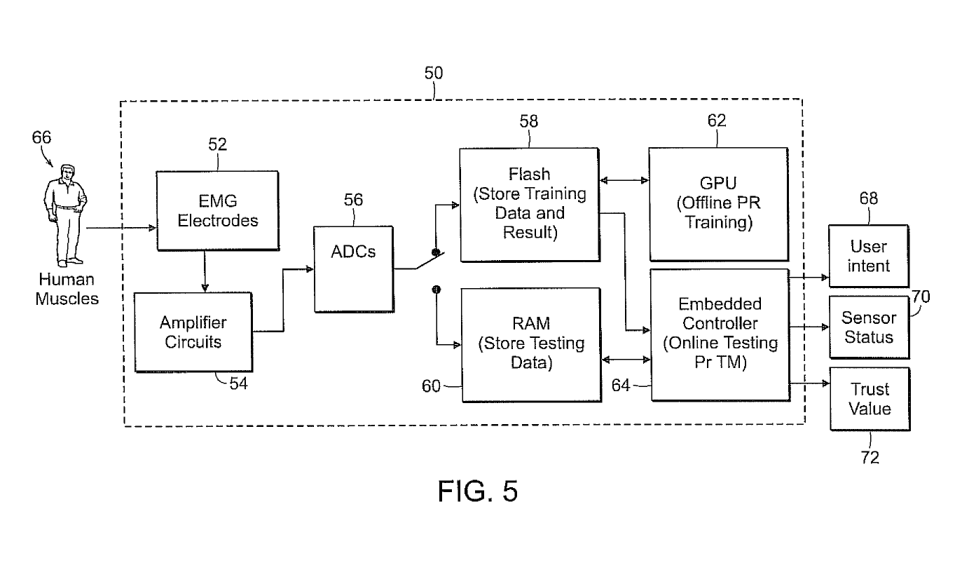

The hardware architecture 50 of the NMI for artificial legs (as shown in FIG. 5) consists of seven components: EMG electrodes 52, amplifier circuits 54, analog-to-digital converters (ADCs) 56, flash memory 58, random access memory (RAM) 58, a graphic processing unit (GPU) 60 and an embedded controller 62. Multiple channels of EMG signals are collected from different muscles on patient's (66) residual limb using EMG electrodes 52. The amplifier circuits 54 are built to make signal polarity, amplitude range, and signal type (differential or single-ended) compatible with the input requirements of ADCs. The outputs of the amplifier circuits 54 are converted to digital format by the ADCs 56 and then stored in the flash memory 58 or the RAM 60.

The embedded hardware works in two modes: training mode and real time testing mode. In the training mode (as discussed above with reference to FIG. 2), a large amount of EMG data are collected and stored in the flash memory. These data are then processed to train the EMG pattern classifier. The pattern recognition (PR) algorithm for the training phase includes complex signal processing and numerical computations, which are done efficiently in a high performance GPU. The parameters of the trained classifier are stored in the flash memory upon completion of the training phase.

The real time testing phase is implemented on the embedded microcontroller 64, including both the PR algorithm and the trust management (TM) algorithm. In the real time testing mode, the EMG signals are sampled continuously and stored in the RAM of the embedded controller. The EMG data are then sent to the trained classifier for a decision to identify the user's intended movement (68) and at the same time each EMG sensor is monitored (70) by an abnormal detector. The trust value (72) of each sensor is therefore, evaluated by the trust manager.

Technical challenges in hardware design are twofold. First, in order to increase the decision accuracy, frequent training computations are often necessary. Such training computations need to be done not only periodically with predetermined time intervals but also whenever the system trust level goes below our predetermined threshold. The training algorithms require intensive numerical computations that requires considerable time in the range of a few minutes to hours on a general purpose computer system. It is important therefore, to substantially speed up this training computation to make the training time of the NMI practically tolerable.

The second challenge is the real time processing of decision making in order to have smooth control of artificial legs. Such real time processing includes signal sampling, AD/DA conversion, storing digital information in memory, executing PR algorithms, periodical trust management, and decision outputs. To meet these technical challenges, we presented a new hardware design incorporating a multi-core GPU and an embedded system with a built-in flash memory.

The neural-machine interface (NMI) employs a high speed, low cost, multi-core GPU (such as the ATI Radeon HD 3650 GPU) for the purpose of speeding up complex PR training computations. The design for the training of the classifier used a NVIDIA 9500GT graphic card that has four multiprocessors with 32 cores working at the clock rate of 1.4 GHz. Each multiprocessor supports 768 active threads giving rise to a total of 3072 threads that can execute in parallel. These threads are managed in blocks. The maximum number of threads per block is 512. The size of the global memory is 1 GB with bandwidth of 25.6 GB/s. 64 KB of the global memory is read-only constant memory. The threads in each block have 16 KB shared memory which is much faster than the global memory because it is cached. The GPU card was connected using the x16 PCI Express bus. Whenever the training computation is triggered, the GPU is called in to perform the training process and store the parameters of trained classifier in the flash memory to be used for real time decision-making.

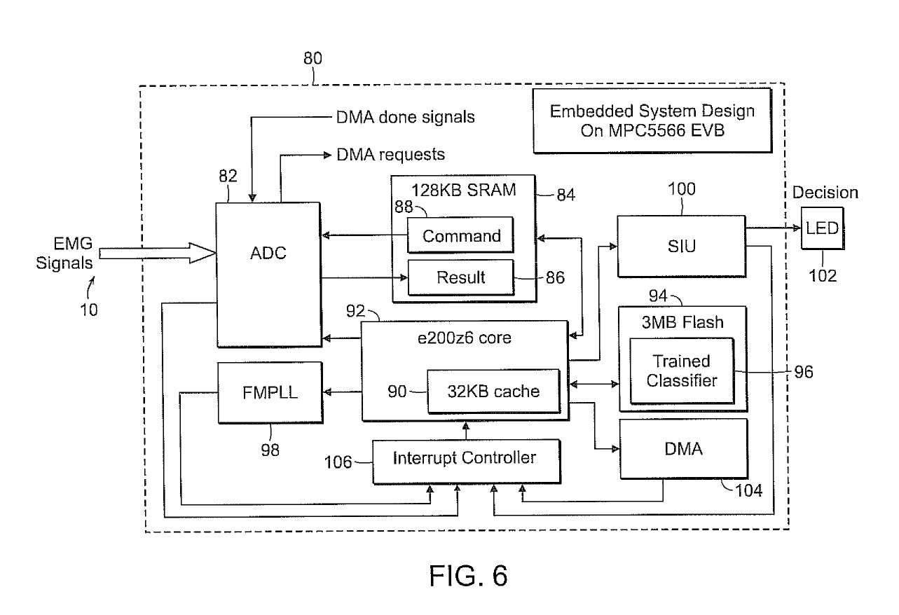

The second part of the hardware design is based on Freescale's MPC5566 132 MHz 32 bits microcontroller unit (MCU) with the Power Architecture. In particular, and as shown in FIG. 6, an embedded controller system 80 in accordance with an embodiment of the invention includes an analog to digital converter unit (82) that provides digital data to a static random access memory unit 84 that receives the result data 86 from the converters 82 and provides commands 88 to the converters 82. The MCU has 40 channels of ADCs with up to 12 bit resolution and two levels of memory hierarchy. The fastest memory is 32 KB unified cache 90 within the e200z6 core 92. The lower level memories include the 128 KB SRAM 84 and a 3 MB flash memory 94 that includes a trained classifier 96. The default system clock of the MCU is 12 MHz. A frequency modulated phase locked loop (FMPLL) 98 generates high speed system clocks of 128 MHz from an 8 MHz crystal oscillator. The direct memory access (DMA) engine 104 transfers the commands and data between the SRAM and the ADC unit without direct involvement of the CPU. Minimizing the intervention from CPU is important for achieving optimal system response. A device system integration unit (SIU) configures and initializes the control of general-purpose I/Os (GPIOs). The real-time results of the embedded system, including the identified user intent, individual sensor status and trust value, are sent to the GPIO pins and displayed by multiple LEDs 102 on the MPC5566 EVB. The ADC 82, FMPLL 98, SIU 100 and DMA 104 all communicate with the core 92 via an interrupt controller 106.

The NMI system was designed to decipher the task transitions between sitting and standing. These tasks are the basic activity of daily living but difficult for patients with transfemoral amputations due to the lack of knee power. During the transition phase, EMG signals are non-stationary. The classifier was designed in the short transition phase. Although it is possible to activate the knee joint directly based on the magnitude of one EMG signal or force data recorded from the prosthetic pylon, unintentional movements of the residual limb in the sitting or standing position may accidentally activate the knee, which in turn may cause a fall in leg amputees. Hence, intuitive activation of a powered artificial knee joint for mode transitions requires accurate decoding of EMG signals for identifying the user's intent from the brain.

For the real time evaluation of the designed pattern recognition algorithm, one male patient with a unilateral transfemoral amputation was recruited. To evaluate the sensor trust algorithm, one male able-bodied subject, free from orthopedic or neurological pathologies, was also recruited. Seven surface EMG electrodes (MA-420-002, Motion Lab System Inc., Baton Rouge, La.) were used to record signals from gluteal and thigh muscles in one side of both subjects. The EMG electrodes contained a pre-amplifier which band-pass filtered the EMG signals between 10 Hz and 3,500 Hz with a pass-band gain of 20. For the able-bodied subject, the monitored muscles included the ipsilateral gluteus maximus (GMA), the rectus femoris (RF), vastus medialis (VM), vastus lateralis (VL), sartorius (SAR), biceps femoris long head (BFL), and semitendinosus (SEM) on the dominant leg of the subject. After the skin was shaved and cleaned with alcohol pads, the EMG electrodes were placed on the anatomical locations. For the amputee subject, the GMA on one side and muscles surrounding the residual limb were monitored. The subject was instructed to perform hip movements and to imagine and execute knee flexion and extension. The EMG electrodes were placed at locations where strong EMG signals may be recorded, and were embedded into a gel-liner system (Ohio Willow Wood, US) for reliable electrode-skin contact. The amputee subject rolled on the gel-liner before socket donning A ground electrode was placed near the anterior iliac spine for both able-bodied and amputee subjects. An MA-300 system (Motion Lab System Inc., Baton Rouge, La.) collected 7 channels of EMG data. The cut-off frequency of the anti-aliasing filter was 500 Hz for EMG channels. All the signals were digitally sampled at a rate of 1000 Hz and synchronized.

The states of sitting and standing were indicated by a pressure measuring mat. The sensors were attached to the gluteal region of the subject. During the weight bearing standing, the recording of the pressure sensors were zero; during the non-weight bearing sitting, the sensors gave non-zero readings.

To evaluate the pattern recognition algorithm, before the real-time system testing, a training session was required in order to collect the training data for the classifier. During the training session, the subject was instructed to perform four tasks (sitting, sit-to-stand, standing, and stand-to-sit) on a chair (50 cm high). For sitting or standing task, the subject was required to keep the position for at least 10 sec. In the sitting or standing position, the subject was allowed to move the legs and shift the body weight. For two types of transitions, the subject performed the transitions without any assistance at least 5 times. During the real-time system evaluation testing, the subject was asked to sit and stand continuously. A total of 5 trials were conducted. In each trial, the subject was required to sit and stand at least five times, respectively. Rest periods were allowed between trials in order to avoid fatigue.

To evaluate the sensor trust algorithm, 13 trials of real-time disturbance detection testing were tested on able-bodied subject. In each trial, motion artifacts were introduced randomly on one EMG electrode in each task phase for four times. To add motion artifacts, the experimenter tapped an EMG electrode with roughly same strength. There were totally 159 times motion artifacts introduced in the whole experiment.

Four classes during the movement initiation phase were considered: sitting, sit-to-stand transition, standing, and stand-to-sit transition. Note that the classes of sitting and standing were not stationary because the subject was instructed to move the legs and shift the body weight in these positions. The output of the classifier was further combined into two classes (class 1: sitting and stand-to-sit transition; class 2: standing and sit-to-stand transition). Four TD features as discussed above and LDA-based classifier were used. Overlapped analysis windows were used in order to achieve prompt system response. For the real-time algorithm evaluation, 140 ms window length and 80 ms window increment were chosen. Two indicators were used to evaluate the real-time performance of EMG pattern classifier: classification accuracy and classification response time. Two types of classification response time were defined: the time delay (RT1) between the moment that the classification decision switched from sitting (0) and standing (1) and the moment that the gluteal region pressure changed from non-zero value (non-weight bearing sitting) to zero value (weight-bearing standing); the time delay (RT2) between the moment that the classification decision switched from standing (1) to sitting (0) and the moment that the gluteal region pressure changed from zero value (weight-bearing standing) to non-zero value (non-weight bearing sitting).

For the real-time evaluation of the abnormal detection and trust management systems, the EMG electrodes recorded EMG signals under the task transitions, unintentional leg movements, as well as disturbances. There were two different states: (1) normal movements (N), including unintentional leg movements and transitions between sitting and standing, the total number of which were 364, and (2) disturbances (D), the total number of which were 159. The detectors detected two types of results: normal (N) or disturbance (D).

For the data sets with motion artifacts, the data in each trial were divided into analysis windows. A state (N or D) was assigned to each window. There were four detection results: (1) Hit (H): Truth=D, Detection=D; (2) False Alarm (F): Truth=N, Detection=D; (3) Miss Detection (M): Truth=D, Detection=N; and (4) Correct no detection (Z): Truth=N, Detection=N.

The performance of the designed detectors were evaluated by the probability of detection (PD) and the probability of false alarm (PFA) as follows:

$$PD = \frac{H}{H + M}$$

$$and$$

$$PFA = \frac{F}{F + Z}$$

The trust values of the sensors were also shown.

The system was implemented on the NMI hardware as discussed above. The offline PR training algorithm, the real time PR testing algorithm, and the real time TM algorithm were all implemented as discussed above. The window length and the window increment were set to 140 ms and 80 ms, respectively. This is because the computation speed of MPC5566 is limited; it takes approximate 80 ms to compute the EMG PR algorithm and to run the abnormal detection/trust evaluation algorithm on data in a 140 ms window using MPC5566. Therefore, the window increment were no less than 80 ms. If the window length is over 120 ms, enlarging the window length does not affect the classification performance but increases the time needed for decision-making, which causes delayed system response.

A parallel algorithm specially tailored to the GPU architecture for the computation intensive part of the PR training algorithm was designed using a Computer Unified Device Architecture (CUDA), which is a parallel computing engine developed by NVIDIA. The GPU was not directly connected to the embedded MCU. Rather, the NVIDIA 9500GT graphic card was plugged into the PCI-Express slot of the PC server to do the training computation. The training results were then manually loaded into the flash memory of the embedded system board for real time testing. The GPU took inputs from 7 EMG channels, each of which had about 10,000 data points. The EMG-data were segmented into analysis windows with 140 ms in length. As a result, each window contained a 140×7 matrix. The training algorithm first extracted 4 TD features from each channel, producing a 28×1 feature vector for each window. The parallel algorithm on the CUDA spawned 7 threads for each window resulting totally 2,800 threads for 400 windows. All these threads were executed in parallel on the GPU to speed up the process. The resultant features were stored in a 28×W matrix, where W is the number of windows. The algorithm then set up K thread blocks, where K is the number of observed motions of the user. Each one of the K thread blocks had 28×14 threads, and a total of K×28×14 threads could execute simultaneously in parallel on the GPU architecture.

To demonstrate the speedup provided by the parallel implementation on the GPU, an experiment was conducted that compared the computation times of the training algorithm on both the GPU system and the fully equipped 3 GHz Pentium 4 PC server.

The real time testing algorithm was implemented on Freescale's MPC5566 evaluation board, integrating both the PR algorithm for user intent identification and the TM algorithm for sensor trust evaluation. The parameters of the trained PR classifier, a 28×4 matrix and a 1×4 matrix, calculated during the training phase by GPU were stored in the built-in flash memory on the MPC5566 EVB in advance. The ADCs sampled raw EMG data of 7 channels at the sampling rate of 1000 Hz continuously. As with the training phase, the EMG data were divided into windows of length 140 ms and increment 80 ms. In every analysis window, 4 TD features were extracted for each individual channel. During the user intent identification process, a 28×1 feature vector was derived from each window and then fed to the trained classifier. After the EMG pattern classification, one movement class out of four was identified. The result was post-processed by the majority vote algorithm to produce a final decision—sitting or standing.

During the sensor trust evaluation process, each EMG sensor was monitored by an individual abnormal detector. Only two of the four TD features (the mean absolute value and the number of slope sign changes) were used to detect motion artifacts. Each abnormal detector monitored the changes of these two TD features to produce a status output for its corresponding sensor: normal or disturbed. The trust level manager then evaluated the trust level of individual sensor based on accumulated disturbance information.

There are two challenges in ensuring a smooth control of artificial legs in a real time embedded system design: precise timing control and efficient memory management. This is due to the speed and memory limitations of the embedded controller. The above disclosed hardware management mechanism was provided on the bare machine of the MPC5566 EVB without depending on any real time OS to avoid unpredictability and delay variations. A circular buffer was designed to allow simultaneous data sampling and decision making. The circular buffer consisted of three memory blocks B1, B2 and B3 that were used to store the ADC sampling data. Each block stored the data sampled in one window increment. An additional memory block, B4, was used as a temporary storage during the computation of PR algorithm and TM algorithm.

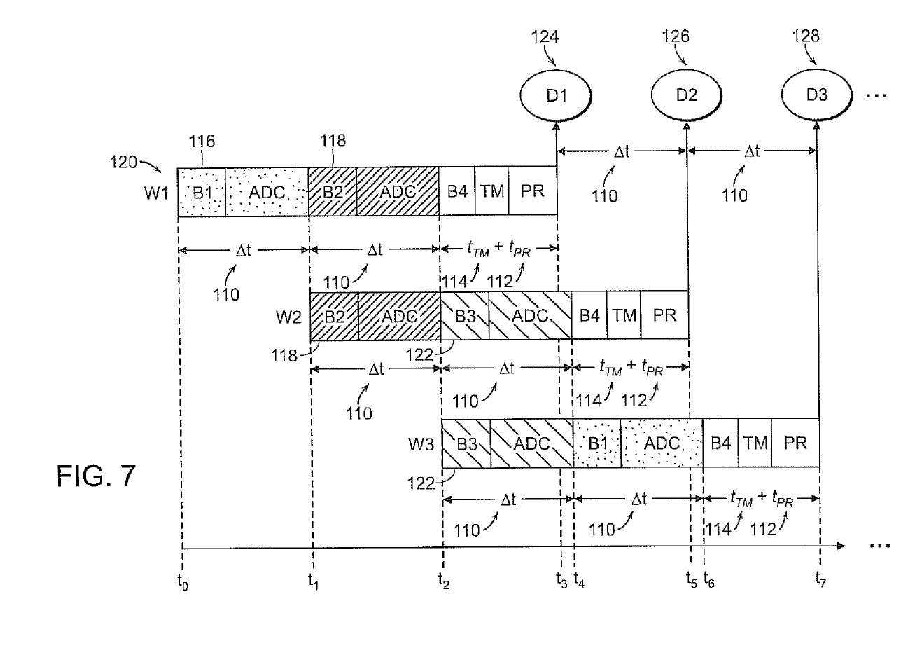

FIG. 7 shows a timing diagram of the control algorithm during the real time testing process. As shown at 110, Δt equals the window increment, tₚᵣ is the execution time of PR algorithm as shown at 112, and t_{TM} is the execution time of TM algorithm as shown at 114. Two conditions need to be satisfied to ensure the smooth control of decision making without delay: (1) t_{TM}+t_{PR}<Δt and (2), t_{w}<2Δt where t_{w} is the window length.

At time t₀ the ADCs begin to sample EMG signals continuously and the digital data are stored in B1 as shown at 116. At time t₁, B1 is filled up and the in-coming data are stored in B2 as shown at 118. At time t₂, the data for the first window W1 120 are available (stored in B1 and B2), and an interrupt request is generated to notify the CPU that the algorithm computation program is ready to run. The algorithm computation then starts.

At the same time, new data keep coming in to be stored in B3 as shown at 122. After the time interval of t_{TM}+T_{PR}, at time t₃, the PR computation and the sensor trust computation of W1 are complete. The first decision 1 is made as shown at 124, identifying user's intent of window W1 whether to sit or stand, and also reporting the status and the trust value of each sensor. At time t₄, B3 is filled up and data for W2 are ready for the algorithm computation again. At this time, B1 is no longer in use so it can be replaced by new sampling data. At time t₅, the decision D2 of window W2 is made as shown at 126. At time t₆, data for W3 (stored in B3 and B1) are available, the algorithm computation for W3 begins. At time t₇, D3 is done as shown at 128 and B2 may be reused.

Using the NMI prototype described above, a real time test was carried out as described above. At the time of the experiment, the trust model focused on abnormal detection and the trust was evaluated at the sensor level. The communication between the trust manager and the classifier was not fully considered. Therefore, to better evaluate our system performance, a two-phase experiment was set up to evaluate the performance of pattern recognition and that of sensor trust management separately. For both phases, the subjects performed transitions between sitting and standing continuously.

During the phase of PR evaluation, there were no motion artifacts manually added. However, the subject's unintentional movements and the movements between the residual limb and prosthetic socket still existed. The movement decisions made by the classification system were displayed on a LED light and a computer monitor in real time. In the experiment, a 5-window majority vote was applied to the decision stream to further eliminate the classification errors. During the phase of sensor trust evaluation, motion artifacts were manually introduced by randomly tabbing an EMG electrode with roughly the same strength and only the sensor status and the sensor trust value were monitored, which were also displayed on a computer monitor. The user intent classification results were ignored during this phase.

During the continuous real-time testing (more than 30 times sit-to-stand transitions and 30 times stand-to-sit transitions), all of the transitions between sitting and standing were accurately recognized. Although the subject moved the legs during the sitting position and shifted the body weight in the standing position, no classification error was observed.

The system classification response time (RT1 and RT2) was calculated by using the pressure data under the gluteal region and is shown in Table 1 below where “+” represents that the classification decision was made after the event (non-weight bearing sitting to weight-bearing standing) and “−” represents that the classification was made before the event (weight-bearing standing to non-weight bearing sitting).

TABLE 1

| RT1 | RT2 |
|---|---|
| +(364 ± 38) ms | −(875 ± 27) ms |

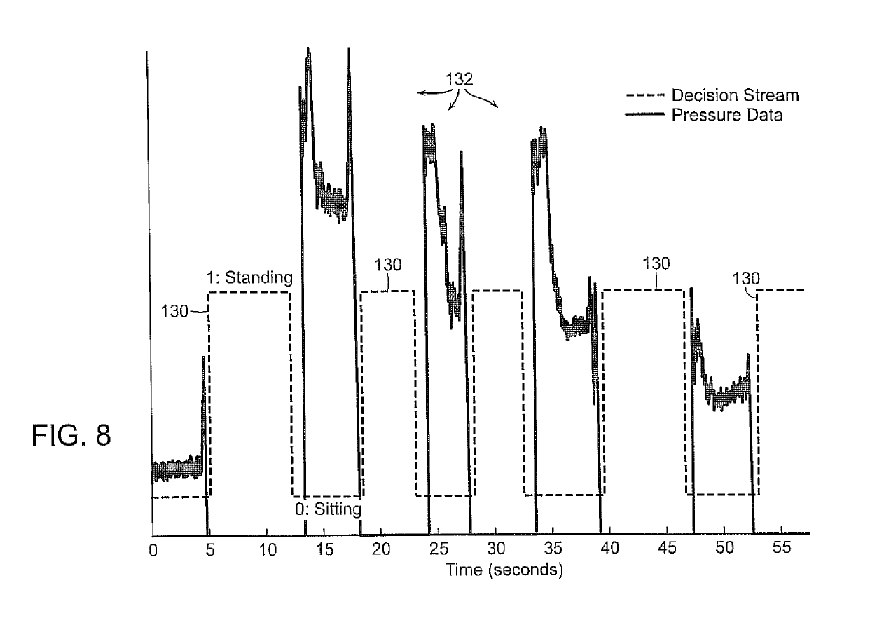

The real-time performance of the above described NMI system in the above representative trial is shown in FIG. 8 where the decision stream is show at 130 and the pressure data is shown at 132. A decision delay of about 400 ms for the sit-to-stand transitions were observed compared to the falling edges of pressure data due to a 5-window majority vote method having been applied. It may be clearly seen that the majority vote post-processing method significantly improved the system accuracy but sacrificed the system response time.

Comparing to the real-time testing results on one able-bodied subject, a similarly high classification accuracy and reasonable system response time were achieved on the patient with transfemoral amputation. The promising real-time performance of the designed NMI prototype demonstrates a great potential to allow the amputee patients to intuitively and efficiently control the prosthetic legs.

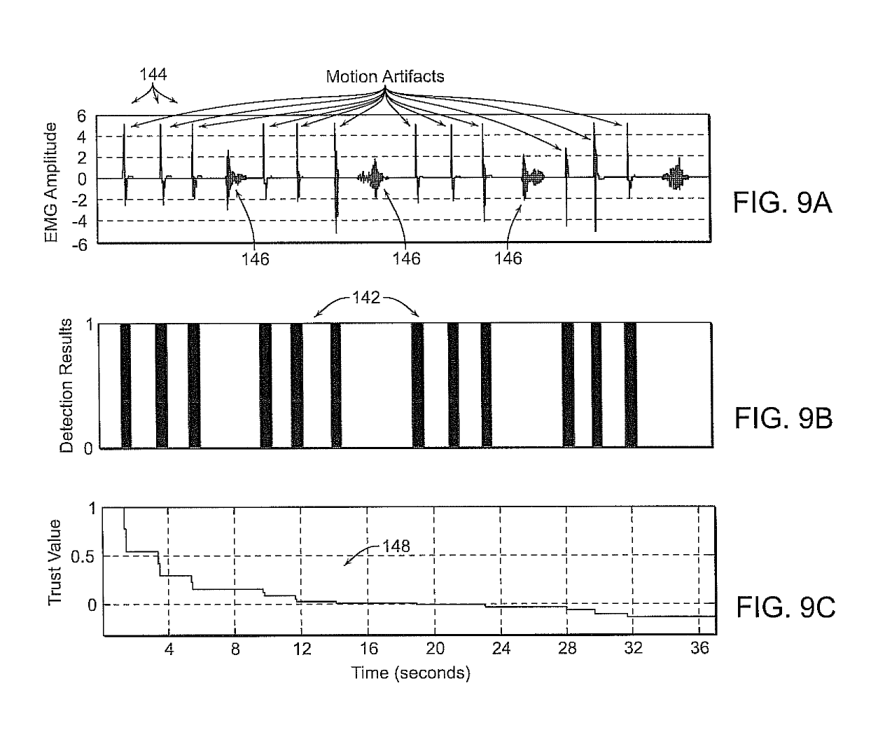

FIGS. 9A-9C show the performance of above discussed trust management method. In particular, the EMG signal disturbed by motion artifacts as shown at 140 in FIG. 9A. The CUSUM detection results are shown in FIG. 9B wherein the bars 142 represent periods in which a motion artifact was detected. As seen in FIGS. 9A and 9B, the CUSUM detector was sensitive to motion artifacts shown at 144, but insensitive to the muscle activity 146 due to the normal leg movements. Additionally, the CUSUM had very small detection delay. The bars 142 were always present immediately after a motion artifact.

FIG. 9C shows at 148 the corresponding trust value, and as shown, the trust value for motion artifacts became gradually reduced when consistent disturbances were detected. In further embodiments, one may monitor whether sensor with non-perfect trust values are consistent with other sensors that have high trust values. By doing so, the sensors that experienced an occasional disturbance and were not damaged may gradually regain the trust. The performance of the CUSUM detector was also evaluated by calculating its detection rate and false alarm rate. During the real time testing experiments, the CUSUM detector achieved 99.37% detection rate and 0% false alarm rate.

Table 2 below shows the measured speedup of the parallel algorithm on the NVIDIA GPU over the PC server for different window sizes.

TABLE 2

| | Window size | | | | |
|---|---|---|---|---|---|
| | 100 | 200 | 400 | 600 | 800 |
| Speedup | 22.98 | 29.50 | 35.94 | 37.16 | 39.21 |

It is clear from Table 2 that the parallel implementation on the GPU gives over an order of magnitude speedup over the PC server. Consider the case where the training time took half hour on a PC server. The same training algorithm takes less than a minute using our new parallel algorithm on the GPU. From an amputee user point of view, training for less than a minute for the purpose of accurate and smooth neural control of the artificial leg is fairly manageable as compared to half hour training every time when training is necessary. Furthermore, the speedup increases as the number of windows increases. As a result, parallel computation of the training algorithm on GPU helps greatly in the NMI design since the larger the number of windows, the higher its decision accuracy will be.

The invention therefore provides a new EMG-based neural-machine interface (NMI) for artificial legs that may be implemented on an embedded system for real time operation. The NMI represents a typical cyber-physical system that tightly integrates cyber and physical systems to achieve high accuracy, reliability, and real-time operation. The cyber-physical system consists of (1) an EMG pattern classifier for decoding the user's intended lower limb movements and (2) a trust management mechanism for handling unexpected sensor failures and signal disturbances. The software may be embedded in hardware platform based on an embedded microcontroller and a GPU to form a complete NMI for real time testing.

Those skilled in the art will appreciate that numerous modifications and variations may be made to the above disclosed embodiments without departing from the spirit and scope of the present invention.

What is claimed is:

1. A neural-machine interface system for providing control of a leg prosthesis, said system comprising a plurality of input channels for receiving electromyographic signals from a subject, feature vector formation means for processing the electromyographic signals, and pattern classification means for identifying the intended movement of the subject's leg prosthesis.

2. The system as claimed in claim 1, wherein said system further includes a sensor trust evaluation system that provides a trust valuation representative of the reliability of a sensor output signal.

3. The system as claimed in claim 2, wherein said trust evaluation system includes a detection system for identifying whether a sensor output includes a disturbance.

4. The system as claimed in claim 1, wherein said system is provided in an embedded controller.

5. The system as claimed in claim 1, wherein said pattern classification means includes a graphics processing unit.

6. The system as claimed in claim 5, wherein said graphics processing unit is a multi-core processing unit

7. The system as claimed in claim 1, wherein said system includes training means for training the system to recognize patterns of electromyographic signals as being associated with different motions.

8. A neural-machine interface system for providing control of a lower limb, said system comprising a plurality of input channels for receiving a plurality of sensor output signal from a subject, processing means for processing the plurality of sensor output signals, pattern classification means for identifying the intended movement of a subject's leg, and sensor trust evaluation means for providing a trust valuation representative of the reliability of each of the plurality of sensor output signals.

9. The system as claimed in claim 8, wherein said trust evaluation system includes a detection system for identifying whether a sensor output includes a disturbance.

10. The system as claimed in claim 8, wherein said system is provided in an embedded controller.

11. The system as claimed in claim 10, wherein said system includes a graphics processing unit.

12. The system as claimed in claim 11, wherein said graphics processing unit is a multi-core processing unit.

13. The system as claimed in claim 8, wherein said system includes training means for training the system to recognize patterns of electromyographic signals as being associated with different motions.

14. A method of providing control of a leg prosthesis, said method comprising the steps of:
receiving a plurality of electromyographic signals at a plurality of input channels;
processing the plurality of electromyographic signals; and
identifying the intended movement of a subject's leg prosthesis.

15. The method as claimed in claim 14, wherein said method further includes the step of providing a trust valuation representative of a reliability of each of the sensor output signals.

16. The method as claimed in claim 15, wherein said step of providing a trust valuation includes the step of identifying whether a sensor output includes a disturbance.

17. The method as claimed in claim 14, wherein said method is provided in an embedded controller.

18. The method as claimed in claim 17, wherein said method is further provided using a graphics processing unit.

19. The method as claimed in claim 18, wherein said graphics processing unit is a multi-core processing unit.

20. The method as claimed in claim 14, wherein said method includes the step of training a system to recognize patterns of electromyographic signals as being associated with different motions.
